(19) United States
(12) Patent Application Publication
(10) Pub. No.: US 2014/0066393 A1
(43) Pub. Date: Mar. 6, 2014

(54) METHOD OF SELECTING CANCER PATIENTS FOR ANTI-ANGIOGENESIS THERAPY IN COMBINATION WITH CHEMOTHERAPY

(71) Applicants: Sherry X. Yang, Ellicott City, MD (US); Sandra M. Swain, Bethesda, MD (US); Seth M. Steinberg, North Potomac, MD (US)

(72) Inventors: Sherry X. Yang, Ellicott City, MD (US); Sandra M. Swain, Bethesda, MD (US); Seth M. Steinberg, North Potomac, MD (US)

(73) Assignee: THE UNITED STATES OF AMERICA, AS REPRESENTED BY THE SECRETARY, DEPT. OF HEALTH AND HUMAN SERVIC, Bethesda, MD (US)

(21) Appl. No.: 14/002,455

(22) PCT Filed: Mar. 1, 2012
(86) PCT No.: PCT/US12/27283

§ 371 Date: Nov. 18, 2013

Related U.S. Application Data
(60) Provisional application No. 61/448,092, filed on Mar. 1, 2011.

(51) Int. Cl. G01N 33/574

(52) U.S. Cl. CPC G01N 33/57415
USPC 514/34; 514/49; 514/449; 435/7.92

(57) ABSTRACT

What is described is a method for identifying a cancer patient that is amenable to anti-angiogenesis therapy in combination with chemotherapy for longer overall survival and/or progression-free survival by measuring the status of tumor cell HER2 expression, p53 expression, and apoptosis or endothelial cell CD31 expression in a tumor sample obtained from the patient prior to treatment. Bevacizumab, an antibody to vascular endothelial growth factor, is a preferred anti-angiogenesis therapy for treating several cancer types including breast cancer. Cancer patients with p53-negative or HER2 negative tumors, especially dual p53 HER2-negative tumors or tumors with low levels of apoptosis or high endothelial cell CD31 expression in the tumor samples are more amenable to anti-angiogenesis therapy with bevacizumab. These tumor characteristics provide a diagnostic method for identifying cancer patients amendable to anti-angiogenesis therapy plus chemotherapy for better treatment outcome.

### CROSS-REFERENCE TO RELATED APPLICATIONS

This application claims benefit under 35 U.S.C. §119(e) of provisional U.S. patent application No. 61/448,092, filed Mar. 1, 2011, the contents of which are incorporated herein by reference.

### TECHNICAL FIELD

This disclosure relates to methods of detecting levels of tumor cell apoptosis, as well as expression of p53 and HER2, which provide methods for identifying a patient that is amendable to anti-angiogenesis therapy in combination with chemotherapy for better treatment outcome.

### BACKGROUND

During the past decade, anti-angiogenesis therapy has evolved as a promising approach to the treatment of cancer. Several clinical trials were conducted to test bevacizumab, a humanized monoclonal antibody to vascular endothelial growth factor A (VEGF-A), as an antiangiogenic agent to treat cancer derived from many tissues, including breast, colorectal, prostate, ovarian, lung, renal cell carcinoma, gastric, pancreatic, and glioblastoma. Four phase III clinical trials have been conducted which demonstrate that the addition of bevacizumab to chemotherapy either as first-line (E2100, AVADO, and RIBBON-1) or second-line (RIBBON-2) therapy prolongs progression-free survival (PFS) and increases the response rate in patients with HER2-negative locally recurrent or metastatic breast cancer (Miller K, et al., 2007, N Engl J Med 357:2666-76; Miles D W, et al., 2010, J Clin Oncol 28:3239-47). However, none of these trials has demonstrated a survival benefit from the addition of bevacizumab to chemotherapy (O'Shaughnessy J, et al., 2010, J Clin Oncol 28:7s: Abstr 1005; Yang SX, 2009, Expert Rev Anticancer Ther 9:1715-25). More recently, two randomized phase III clinical trials evaluated neoadjuvant bevacizumab and chemotherapy in the treatment of early stage and locally advanced HER2-negative breast cancer. The addition of bevacizumab significantly increases pathological complete response in the breast and clinical responses (complete and partial). Most recently, AVEREL randomized phase III trial evaluated combining bevacizumab and trastuzumab plus chemotherapy in HER2-positive locally recurrent or metastatic breast cancer. The addition of bevacizumab to trastuzumab plus chemotherapy improved progression-free survival and objective response rate without reaching statistical significance by investigator assessment. It significantly increases the objective response rate and progression-free survival after stratification, but not overall survival by the Independent Review Committee (SABCS 2011).

For these reasons, identification of certain characteristic factors of a tumor that are associated with or potentially predict benefit for appropriate identification of patients for personalized treatment has become a key issue of anti-angiogenesis therapy and an intense area of clinical and translational research. This is important also because anti-angiogenesis therapy is very expensive ($100,000 per year per patient) and can sometimes cause severe undesirable side effects. Thus it is imperative to develop and validate methods that could associate certain characteristics of a tumor sample with treatment outcome, especially survival.

### SUMMARY

One aspect of the disclosure is a method for identifying a cancer patient who is amenable to an anti-angiogenesis therapy plus chemotherapy by measuring p53 expression and levels of apoptosis in a tumor sample obtained from the cancer patient prior to initiating the combination treatment. In a further embodiment of this method, the anti-angiogenesis therapy comprises administration of an anti-angiogenic agent selected from the group consisting of an anti-vascular endothelial growth factor (VEGF) monoclonal antibody and a small-molecule kinase inhibitor that is specific to a VEGF receptor, Raf, a platelet-derived growth factor receptors (PDGFR), or Kit. Preferred small-molecule kinase inhibitors are sunitinib, sorafenib, or pazopanib. The most preferred anti-angiogenesis therapy in several cancer types comprises administration of bevacizumab.

Another embodiment of the disclosure further comprises administration of a chemotherapeutic agent, including a taxane (paclitaxel or docetaxel), an anthracycline (doxorubicin or epirubicin), cyclophosphamide, capecitabine, tamoxifen, letrozole, carboplatin, gemcitabine, cisplatin, erlotinib, irinotecan, fluorouracil, or oxaliplatin.

Another aspect of the disclosure is identifying cancer patient for anti-angiogenesis therapy with bevacizumab and a chemotherapeutic agent, wherein the cancer patient has a solid tumor derived from the breast, prostate, ovarian, renal cell carcinoma, lung, gastric, pancreas, glioblastoma, and colorectal. The preferred cancer patient for angiogenesis therapy is a breast cancer patient. A preferred embodiment is a method of identifying a breast cancer patient for treatment with bevacizumab and at least a chemotherapeutic agent. A preferred chemotherapeutics based therapy comprises administration of capecitabine, paclitaxel, an anthracycline, or docetaxel.

Another aspect of the disclosure is a method for identifying a patient amenable to combination treatment with bevacizumab and a chemotherapeutic agent or a chemotherapy regimen by measuring the status of p53 expression and apoptosis, and additionally measuring the status of HER2 expression in a tumor sample obtained from the patient. Another embodiment of the disclosure comprises identifying a patient amenable to bevacizumab and a chemotherapeutic agent or a chemotherapy regimen based on the level of apoptosis, preferably wherein level of apoptosis in the sample is low as measured by an appropriate method as described herein. Another embodiment of the disclosure comprises identifying a patient amenable to bevacizumab and a chemotherapeutic drug or a chemotherapy regimen based on the level of endothelial CD31 expression in the tumor vasculature, preferably wherein level of CD31 expression is high as measured by an appropriate method as described herein. Another embodiment of the disclosure comprises identifying a patient amenable to bevacizumab and a chemotherapeutic agent or a chemotherapy regimen based on the status of p53 expression in a tumor sample obtained from the patient, preferably wherein the patient is identified based on the status of p53 expression and HER2 expression in the tumor sample, most preferably wherein p53 and HER2 expression are both negative as measured by an appropriate method as described herein.

Another aspect of the disclosure is a method for identifying a breast cancer patient amenable to the combination treatment, comprising measuring the status of certain characteristics in a tumor sample obtained from the patient, and identifying the patient for anti-angiogenic treatment based on the status of certain characteristics in the tumor sample, wherein the anti-angiogenic therapy is neoadjuvant bevacizumab plus chemotherapy. Another embodiment of the disclosure is the method wherein the status of certain characteristic factors of a tumor sample are HER2 expression, p53 expression, apoptosis, or CD31 expression preferably wherein the patient is identified based a low level of apoptosis, p53 expression negative, HER2 expression negative or high expression of endothelial CD31 in the tumor sample as measured by an appropriate method as described herein. Another embodiment of the disclosure is the method, wherein the chemotherapy is based on administration of docetaxel/doxorubicin or paclitaxel.

Another aspect of the disclosure is a method for identifying a breast cancer patient amenable to the combination treatment, comprising measuring levels of certain characteristics in a tumor sample obtained from the patient, and identifying the patient for anti-angiogenic therapy based on the status of HER2 expression, p53 expression, apoptosis or endothelial cell CD31 expression in the tumor sample, wherein the cancer is a primary breast cancer with early or locally advanced stage and inflammatory breast cancer before surgery; a locally recurrent breast cancer, or a metastatic breast cancer, and wherein the anti-angiogenic therapy comprises administration of bevacizumab plus chemotherapy.

Another aspect of the disclosure is a method for identifying a breast cancer patient for the combination treatment, comprising measuring levels of certain characteristics in a tumor sample from the patient, and identifying the patient for anti-angiogenic therapy based on the status of HER2 expression, p53 expression, apoptosis or endothelial cell CD31 expression in the tumor sample, and wherein the cancer is early operable breast cancer, and wherein the anti-angiogenic therapy comprises administration of bevacizumab.

Another aspect of the disclosure is a method of diagnosing a tumor sample in a cancer patient, the method comprising obtaining a tumor sample from the patient; providing a test cell population from the tumor sample; measuring the number or percentage of p53-positive tumor cells and apoptotic cells in the test cell population; and measuring the levels (staining index) of endothelial cell CD31 expression in blood vessels near the tumors of a patient tumor sample. The number or percentage of p53-positive cells and apoptotic cells measured in this way indicates that the tumor is amenable to a combination of anti-angiogenesis therapy and chemotherapy. A further embodiment of this method further comprises a step of measuring the number or percentage of HER2-positive cells in the tumor cells of the test cell population, wherein the levels or percentage of p53-positive cells, apoptotic cells and HER2-positive cells indicates that the tumor is amenable to a combination of anti-angiogenesis therapy and chemotherapy. Preferably, the tumor is determined to be amenable to the combination therapy when the tumor cells of the patient from the test cell population are p53-negative, have low levels of apoptosis and high level of endothelial cell CD31 expression, and are HER2-negative. Most preferably, the tumor is determined to be amenable to the combination therapy when less than ten percent (10%) of the tumor cells are p53-positive, or when the level of apoptosis in the test tumor cell population is less than 2%, or when the HER2 score of the test tumor cell population is 0 or 1+ or 2+, in which the HER2 gene is non-amplified by FISH analysis, or the level of endothelial cell CD31 expression is more than 25 by staining index. In a further embodiment of this method, the anti-angiogenesis therapy comprises administration of an anti-angiogenic agent selected from the group consisting of an anti-vascular endothelial growth factor (VEGF) monoclonal antibody and a small-molecule kinase inhibitor that inhibits activation of VEGF receptors, Raf, platelet-derived growth factor receptors (PDGFR), or Kit. Preferably the anti-angiogenesis therapy comprises administration of bevacizumab, and the chemotherapy comprises administration of a chemotherapeutic agent selected from the drug classes consisting of a taxane (paclitaxel or docetaxel), an anthracycline (doxorubicin or epirubicin), cyclophosphamide, capecitabine, tamoxifen, letrozole, carboplatin, gemcitabine, cisplatin, erlotinib, irinotecan, fluorouracil, and oxaliplatin. In another further embodiment of the method, the cancer patient is selected from the group of cancer patients having solid tumors derived from breast, prostate, ovary, renal carcinoma, lung, gastric, pancreas, glioblastoma, and colorectal tissue. Preferably, the cancer patient has a solid tumor derived from breast tissue, and the chemotherapy regimens contain capecitabine, paclitaxel, an anthracycline, docetaxel, cisplatin, carboplatin, cyclophosphamide, oxilaplatin, irinotecan, 5-flurouraicil, gemcitabine topotecan or vincristine.

### BRIEF DESCRIPTION OF THE DRAWINGS

FIG. 1. Overall survival (OS) comparing apoptosis-low group with apoptosis-high group (A) and p53-negative group with p53-positive group (B), or progression-free survival comparing HER2-negative group to HER2-positive group (C) and comparing the group with high level of endothelial cell CD31 expression with that with low level of endothelial cell CD31 expression (D).

FIG. 2. Overall survival (OS) in patients treated with bevacizumab plus chemotherapy according to HER2 and p53 status. OS comparing patients with p53-negative tumors to those with p53-positive tumors in HER2-negative group (A) and HER2-positive group (B).

### DETAILED DESCRIPTION OF ILLUSTRATIVE EMBODIMENTS

### Anti-Angiogenesis Therapy

Anti-angiogenesis drugs don't attack cancer cells directly. Instead, they target the blood vessels the cancer cells need to survive and grow, and so may help prevent the tumor cells from growing. They may also make large tumors shrink if their blood supply is cut off. Cancer cells use a number of different pathways to promote blood vessel growth. Different drugs may work at different steps in these pathways.

One of the most important proteins in new blood vessel growth is the vascular endothelial growth factor A (VEGF). This protein is not made in large amounts by normal cells, but some cancer cells make it and release it into the extracellular matrix which surrounds them. VEGF then attaches to the proteins (called the VEGF receptors, or VEGFRs) on the surface of nearby endothelial cells or tumor cells. The attachment signals the endothelial cells' control centers to start growing and forming new blood vessels.

Many of the anti-angiogenesis drugs used today attack the VEGF pathway. Bevacizumab (AVASTIN®) is a monoclonal antibody that binds to VEGF and keeps it from reaching the VEGF receptors. Other drugs, like sunitinib (SUTENT®), sorafenib (NEXAVAR®), and pazopanib are small molecules that inhibit the activation of VEGF receptors, PDGFRs, and Kit, and decreasing the growth of new blood vessels. Yet other drugs target downstream signalling proteins in the pathway to the nucleus, including Raf.

The U.S. Food and Drug Administration (FDA) has approved bevacizumab (Avastin®) for use with chemotherapy drugs to treat colorectal cancer that has spread to other parts of the body, some non-small cell lung cancers, and some breast cancers that have spread to other parts of the body. However, in 2011 the FDA revoked the indication of bevacizumab for metastatic HER2-negative breast cancer due to the lack of survival benefit for the patient population and some severe side effects. Yet, it is clear that subsets of breast cancer patients benefit from bevacizumab treatment.

Anti-angiogenesis therapy is being tested in Phase II and III clinical trials for the following cancers: breast cancer, colorectal, esophageal cancer, gastrointestinal stromal tumors (GIST), kidney (renal cell) cancer, leukemia, liver (adult primary) cancer, lymphoma, melanoma, multiple myeloma, non-small cell lung cancer (NSCLC), ovarian epithelial cancer, pancreatic cancer, prostate cancer, glioblastoma, and stomach (gastric) cancer.

Some chemotherapy drugs already in use to treat cancer have been found to affect blood vessel growth, as well as specific anti-angiogenic agents. Such drugs include a taxane (paclitaxel or docetaxel), an anthracycline (doxorubicin or epirubicin), cyclophosphamide, capecitabine, tamoxifen, letrozole, carboplatin, gemcitabine, cisplatin, erlotinib, irinotecan, fluorouracil, and oxaliplatin.

Anti-angiogenesis drugs such as bevacizumab are sometimes combined with chemotherapy, other targeted drugs, or radiation. When used along with chemotherapy to treat certain cancers, bevacizumab helps patients with certain types of cancer live longer.

The methods disclosed herein are helpful to identify cancer patients that are amenable to anti-angiogenic therapy, more preferably therapies involving bevacizumab, sunitinib, sorafenib, and pazopanib, most preferably to therapies involving bevacizumab.

The methods disclosed herein are helpful to identify cancer patients who would be amenable to anti-angiogenic therapy involving bevacizumab and chemotherapy drugs, including paclitaxel, docetaxel, doxorubicin, epirubicin, cyclophosphamide, capecitabine, tamoxifen, letrozole, carboplatin, gemcitabine, cisplatin, erlotinib, irinotecan, fluorouracil, and oxaliplatin. Therapies involving bevacizumab and chemotherapy drugs can be first or second line treatment for locally recurrent or metastatic breast cancer, or as neoadjuvant or adjuvant therapy for early stage and locally advanced breast cancer.

The methods disclosed herein can be applied to breast cancer, colorectal, esophageal cancer, gastrointestinal stromal tumors (GIST), kidney (renal cell) cancer, leukemia, liver cancer, lymphoma, melanoma, multiple myeloma, non-small cell lung cancer (NSCLC), ovarian epithelial cancer, pancreatic cancer, prostate cancer, and stomach (gastric) cancer, preferably to cancers involving solid tumors of the breast, kidney, NSCLC, ovaries, pancreatic cancer, prostate cancer, and gastric, colorectal, and glioblastoma, most preferably breast cancer. The breast cancer can be early, inflammatory, locally advanced, metastatic, locally recurrent, or at any stage, including stage I, IIA, IIB, IIIA, IIIB, IIIC, and IV.

### Characteristics of the Tumor Sample

The disclosure involves identification of cancer patients for the combination treatment by examining expression of certain characteristics of a tumor sample obtained from the patient. These include p53, tumor apoptosis (TUNEL), proliferation (Ki67), estrogen receptor (ER), VEGF-A, pVEGF, VEGFR2 activation (VEGFR2-Y951), human epidermal growth factor receptor 2 (HER2), PDGFR-beta, endothelial cell CD31, serum VCAM-1 or microvessel density (MVD), preferably VEGFR2, p-VEGFR2, and VEGF-A, pVEGF, apoptosis, Ki67, HER2, CD31, and p53, most preferably tumor cell apoptosis, HER2, p53, and endothelial cell CD31 alone or in combination.

Immunohistochemical staining signal can be analyzed quantitatively, e.g., with the assistance of the Automated Cellular Imaging System (ACIS; ChromaVision Medical Systems Inc, San Juan Capistrano, Calif., or manually under the microscope.

### Apoptosis

The disclosure includes a method for identifying patients for the combination treatment by measuring apoptosis alone or in combination with other characteristics of the tumor sample. Apoptosis is a process of programmed cell death characteristic of cell morphology changes, chromatin condensation, DNA fragmentation, and cell death. The process of apoptosis is controlled by a diverse range of cell signals including nutrient deprivation and hypoxia. The latter are largely dependent on the status of blood supply and tissue angiogenesis.

Apoptosis can be measured by using a modified terminal deoxynucleotidyl transferase (TdT)—mediated dUTP nick-end labeling (TUNEL) assay (R&D Systems, Minneapolis, MN. This assay measures the fragmented DNA in apoptotic cells. DNA breaks at 3′-DNA ends can be labeled with biotinylated nucleotides catalyzed by TdT. An avidin-conjugated horseradish peroxidase specifically binds to the biotinylated DNA fragments (Vector Laboratories Inc, Burlingame, Calif.) and generates a brown precipitate in the presence of diamenobezidine. Nuclease-treated breast cancer specimen section can be used as a positive control, and the same tumor sample section without nuclease treatment can be used as a negative control.

Tumors are scored positive for apoptosis when greater than 0.5%, 1%, 2%, 3%, 4%, 5%, 6%, 8%, or 10% are positive for the apoptosis marker, preferably greater than 1%, most preferably greater than 2%.

p53

The disclosure includes a method for identifying patients for combination treatment by measuring the status of p53 expression, alone or in combination with other characteristics. p53 is a nuclear transcription factor encoded by the TP53 gene located on the short arm of chromosome 17 (17p13.1). It is a tumor suppressor which regulates the cell cycle and plays a critical role in the regulation of apoptosis, genetic stability, or inhibition of angiogenesis (Xu H, et al., 2001,. Biotechnol Annu Rev 7:131-64). Wild-type p53 protein has a short half-life with low intracellular levels. However, stabilization of p53 protein in the absence of a stimulus such as DNA damage is a hallmark of loss of function secondary to a mutation, or interaction with viral or cellular oncoproteins. p53-positive tumors versus p53-negative tumors were associated with poor prognosis in breast cancer (Silvestrini R, et al., 1993, J Natl Cancer Inst 85:965-70; Beenken S W, et al., 2001, Ann Surg 233:630-8; Dumontet C, et al., 2010, Clin Cancer Res 16: 3988-97). The dysfunction of p53 contributes to angiogenic switch during tumorigenesis through the amplification of hypoxia-inducible factor 1 (HIF-1) dependent responses to hypoxia. Furthermore, p53-deficient animal models were less responsive than wild-type p53 models to anti-angiogenic and cytotoxic combination therapy.

p53 expression can be measured in a variety of ways known to the person of ordinary skill in the art. For example p53 can be detected by immunohistochemistry using a mouse monoclonal antibody (clone DO7, Vector Laboratories, Inc., Burlingame, Calif.) that recognizes both mutant and wild-type forms of the protein in 1: 50 dilution (Turpeinen M, et al., 2002, Biochem Biophys Res Commun 293: 850-6). Breast cancer cell line MDA-MB-231 (p53-mutant), and a colon cancer specimen that express p53 can be utilized as positive controls. The isotype control mouse immunoglobulins can be used as negative controls. After inactivating the endogenous peroxidase activity and blocking the nonspecific antigen binding sites, the tissue sections can be incubated with the primary antibody, and this binding to its antigenic sites in the tissue sections can be amplified with the use of species-appropriate biotinylated secondary antibodies supplied by the Vectastain Elite ABC kits (Vector Laboratories), and incubating with avidin-peroxidase conjugate (Vectastain Elite ABC kits), followed by 3,3′-diaminobenzidine (Sigma, St. Louis, Mo.). Slides can be counterstained with Mayer's hematoxylin (BioGenex Laboratories) or methyl green (R&D Systems, Minneapolis, MN) and coverslipped with Permount.

Nuclear p53 staining was scored as positive when staining was evident in greater than 1%, 2%, 5%, 7%, 10% 15%, 20%, or 25% of malignant nuclei stained in the tissue section, preferably greater than 5%, 10% or 20%, most preferably greater than 10%. (For the 10% cutoff, when fewer than 10% of the nuclei were stained, the slide was scored as negative.) For statistical analysis, marker scores may be categorized for analysis purposes as positive versus negative for p53. Also, see Table I, below for expression frequencies of this marker.

### HER2, and Other Characteristics

The disclosure includes a method for identifying patients for combination treatment by measuring the status of HER2 expression, alone or in combination with other characteristics. HER2 is encoded by the ERBB2 gene. It activates multiple cellular signaling pathways that are involved in cellular proliferation and survival, and increases VEGF protein synthesis. The latter is regulated via activation of the mTOR/p70S6K cap-dependent translation pathway in human breast cancer cells. Overexpression of HER2 has been shown to correlate with the increased angiogenesis and VEGF-A expression in cancer cells (Kumar R, et al., 2001, Semin Oncol 28: 27-32).

The disclosure includes a method for identifying patients for combination treatment by measuring the status of endothelial cell CD31 expression, alone or in combination with other characteristics. CD31, cluster of differentiation 31, is a platelet endothelial cell adhesion molecule (PECAM1) that is encoded by the PECAM1 gene located on chromosome 17.^{[1]} It is found on the surface of many cell types primarily endothelial cells and is traditionally used for the identification of blood vessels. Previously, we found that a higher endothelial cell CD31 expression in the tumor vasculature at both gene transcription and translation levels was significantly associated with the response to bevacizumab treatment followed by bevacizumab plus chemotherapy. Furthermore, a significant decrease in CD31 expression was observed in patient tumors treated with bevacizumab alone (Yang et al. Clin Cancer Res 2008; 14: 5893-5899). A down-regulation of CD31 after bevacizumab treatment was found in an in vivo lung tumor xenograft model (Inoue et al., Mol Ther 2007;15:287 -94). These data suggest that VEGF-A regulates the level of CD31 expression in endothelial cells and may play a role in the combination treatment with bevacizumab and chemotherapy.

CD31 expression was measured by immunohistochemistry using a specific CD31 antibody at a dilution of 1 to 20 (Clone JC70A, DAKO). The levels of expression were quantitatively analyzed with the assistance of an Automated Cellular Imaging System (DAKO) in which CD31 in vessels in three tumor areas were scored using a 60' tool or a free- scoring tool. Staining index was used to report the levels of CD31 expression, which the index is derived from the percentage of staining multiplied by staining intensity after subtracting the tissue readouts of the corresponding negative control per 100.

Baseline plasma VEGF and serum VCAM-1 were measured by enzyme-linked ImmunoSorbent assay (ELISA; R & D Systems). See Table I for details about the cutoff frequencies used for scoring the circulating markers.

HER2, estrogen receptor (ER), VEGF-A, and VEGFR2-Y951 expression in tumor cells can be measured by immunohistochemistry and scored as described above for CD31 but scoring 6 area of tumor cells. PDGFR-beta expression in tumor stroma and tumor cells is measured by immunohistochemistry and scored as described above for CD31. See Table I, below, for details about the cutoff frequencies useful in scoring these markers. For example, the cutoff for HER2 overexpression were positive when scored at 2+ or 3+ and were negative when scored 0 or 1+ or 2+ but HER2 gene non-amplified by FISH; most preferably scored 3+ in more than 30% of invasive tumor cells or fluorescence in situ (FISH) amplified (ratio of HER2 to CEP17 of ≧2). The cutoff for ER-positive cells is at least 10%, most preferably at least 1%.

### Ki67 Proliferation

The disclosure includes a method for identifying patients for anti-angiogenesis therapy by measuring the status of proliferation (Ki67), alone or in combination with other characteristics. Ki67 is a nuclear protein that is associated with the cellular proliferation, which is widely used as a proliferation marker, especially as a tumor proliferation marker. It is present in all active phases of the cell cycle including G1, S, G2, and mitosis, but is absent from the resting cells. Levels of Ki67 are low in G1 and S phases and highest in mitosis. Low Ki67 as compared with high Ki67 has been found to be associated with longer disease-free survival (DFS) and overall survival (OS) in node-positive breast cancer after adjuvant chemotherapy (Hugh J, et al., 2009, J Clin Oncol 27: 1168-76).

Each slide was scored based on the percentage of positively stained malignant nuclei. The following ranges were used: 0% to 10%, 21% to 30%, and 31% to 100%. Marker scores were categorized for analysis purposes as 31% to 100% versus 0% to 30% for Ki-67. Also, see Table I for expression frequencies of this characteristic factor of the tumor.

### EXAMPLES

Twenty-one previously untreated patients with inflammatory breast cancer (IBC) and locally advanced breast cancer (LABC) were entered into a pilot trial at the National Cancer Institute (NCI, Bethesda, Md.), and treated with neoadjuvant bevacizumab for one cycle, followed by six cycles of bevacizumab plus docetaxel-doxorubicin chemotherapy before surgery. Given the implications of p53, HER2, tumor apoptosis (TUNEL), proliferation (Ki67), estrogen receptor (ER), VEGF-A, pVEGF, VEGFR2 activation (VEGFR2-Y951) and microvessel density (MVD) on tumor progression, angiogenesis, or responses to anti-angiogenic therapy as well as to chemotherapy, evaluation of these tumor characteristics was designed and included in the study protocol (Wedam S B, et al., 2006, J Clin Oncol 24:769-77; Yang S X, et al., 2008, Clin Cancer Res 14: 5893-99). The association of OS and progression-free survival (PFS) was assessed in relation to baseline expression of p53, HER2, ER, VEGF-A, VEGFR2-Y951, tumor apoptosis, proliferation, pVEGF, and MVD, as well as age, grade, and TNM stage.

In brief, the study was approved by the Institutional Review Board of the NCI. Eligible patients signed informed consent which included tissue biopsy and research use of collected tissue in compliance with federal and institutional guidelines. Twenty patients with IBC and one with LABC were enrolled from October 2001 to August 2004. Tumor biopsies were taken by mammotome on the breast tumor area or by either 16 or 18 gauge needles on ipsilateral lymph nodes. The biopsies were immediately fixed in formalin and subsequently sent to the Laboratory of Pathology, National Institutes of Health Clinical Center, for paraffin-embedding and tumor confirmation. Patients were treated with one cycle of bevacizumab at 15 mg/kg followed by six cycles of bevacizumab plus doxorubicin at 50 mg/m2 and docetaxel at 75 mg/m2 every three weeks prior to surgery and/or locoreginal therapy. Patients further received eight cycles of bevacizumab after surgery and those with hormone receptor-positive tumors also received tamoxifen or an aromatase inhibitor treatment. No patients received trastuzumab therapy.

p53, Ki67, VEGF-A, VEGFR2-Y951, PDGFR-beta, CD31, and MVD were examined on formalin-fixed, paraffin-embedded biopsy sections using a standard avidin-biotin-peroxidase complex indirect immunoperoxidase procedure, and quantitatively analyzed with the assistance of a digital imaging system as previously described (Tan A R, et al., 2004, J Clin Oncol 22: 3080-90; Yang et al., 2005, Clin Cancer Res 11:6226-32). Antibodies used were appropriately validated prior to their application to biopsy sections. HER2 status and ER status were determined on the diagnostic biopsy sections by the Laboratory of Pathology. Tumor apoptosis was examined by the terminal deoxynucleotidyl transferase (TdT)—mediated dUTP nick-end labeling (TUNEL) assay; and pVEGF was measured by human VEGF enzyme linked immunosorbent assay (ELISA).

The cutoffs for HER2, ER, and p53 have been described previously (McCarthy N J, et al., 2002, Clin Cancer Res 8: 3857-62; Ince W L, et al., J Natl Cancer Inst 97:981-89), and the cutoffs for Ki67 labeling index, apoptosis index, VEGF-A, pVEGF, and VEGFR29-Y951 were chosen near the median of each characteristic factor of the tumor (Table 1; Abbreviations: ER, estrogen receptor, HER2, human epidermal growth factor receptor 2, MVD, microvessel density, N/A, not applicable, No, number, pVEGFR2, phosphorylated vascular endothelial growth factor receptor 2, pVEGF, plasma vascular endothelial growth factor, VEGF-A, vascular endothelial growth factor A.).

TABLE 1
Expression frequencies of baseline markers.

| Marker | Median (range) | Cutoff | No. (%) |
|---|---|---|---|
| p53 | 6.7 (0-95.7) | ≧10 | |
| | | Negative | 11 (52) |
| | | Positive | 10 (48) |
| HER2 | N/A | ≧2+ | |
| | | Positive | 12 (57) |
| | | Negative | 9 (43) |
| ER | N/A | ≧10 | |
| | | Positive | 11 (71) |
| | | Negative | 6 (29) |
| VEGF-A | 1.2 (0-39.4) | ≧2 | |
| | | Low | 11 (55) |
| | | High | 9 (45) |
| sVEGF | 66.3 (11.03-464.3) | ≧70 | |
| | | Low | 11 (58) |
| | | High | 8 (42) |
| pVEGFR2-Y951 | 21.78 (0.77-76.76) | >20 | |
| | | Low | 10 (50) |
| | | High | 10 (50) |
| MVD | 239.3 (1-441.6) | ≧200 | |
| | | Low | 9 (45) |
| | | High | 11 (55) |
| Ki67 | 29.1 (5.5-84) | >30 | |
| | | Low | 13 (65) |
| | | High | 7 (35) |
| Apoptosis | 1.5 (0-12.8) | >2 | |
| | | Low | 12 (60) |
| | | High | 8 (40) |
| PDGFR-beta | 1.9 (0.06-36.6) | >2 | |
| | | Low | 9 (50) |
| | | High | 9 (50) |
| CD31 | 25.3 (6.2-48.8) | >25 | |
| | | Low | 9 (50) |
| | | High | 9 (50) |
| sVCAM-1 | 560.5 (142.0-1577.9) | >550 | |
| | | Low | 9 (50) |
| | | High | 9 (50) |

In brief, 2+and 3+levels of HER2 expression, but not 0, 1+, and 2+, but HER2 non-amplified by FISH analysis, were scored as positive. ER and p53 were scored as positive if 10% or greater of malignant nuclei were stained.

The probability of OS or PFS as a function of time was estimated for each of certain characteristics by the Kaplan-Meier method. The statistical significance of the differences in the associated comparisons was determined using the log-rank test. Hazard ratios (HR) and 95% confidence intervals (CI) were determined using Cox proportional hazards regression analysis. Follow-up on living patients was current as of July, 2008, and used to determine the duration of OS and PFS. OS was determined from the on-study date to the date of death from any cause or last follow-up. PFS was calculated from the on-study date to the date of disease progression; the follow-up was censored if the patients went off study for reasons other than progression. The factors which were associated with outcome, with a univariate p-value of approximately 0.10 or less, as well as age and stage, were subsequently evaluated for their joint association with OS or PFS by a multivariable Cox proportional hazards model. All patients had consistently high grade tumors. A backward selection algorithm was used to determine the parameters to include in the final model. All p-values were two-tailed, reported without adjustment for multiple comparisons, and considered to be statistically significant at p<0.05.

### Patients, Follow-Up, and Expression of Tumor and Angiogenic Markers.

The median OS was 65.9 months while the median PFS was 17.4 months. Table 1 (above) lists the expression frequency of p53, HER2, Ki67, apoptosis, ER, VEGF-A, pVEGF, VEGFR2-Y951, PDGFR-beta, CD31, sVCAM-1 or MVD. Expression of p53, HER2 or ER was 48%, 43% or 29%, consistent with the previous findings in studies with breast cancer and IBC (Hance K W, et al., 2005, J Natl Cancer Inst 97:966-75; Van den Eynden G G, et al., 2004, Breast Cancer Res Treat 85:13-22. Baseline Ki67 proliferation index ranged from 5.5 - 84% (median 29.1%), and apoptosis index ranged from 0 to 12.8% (median 1.5%).

### Marker Status and Overall Survival.

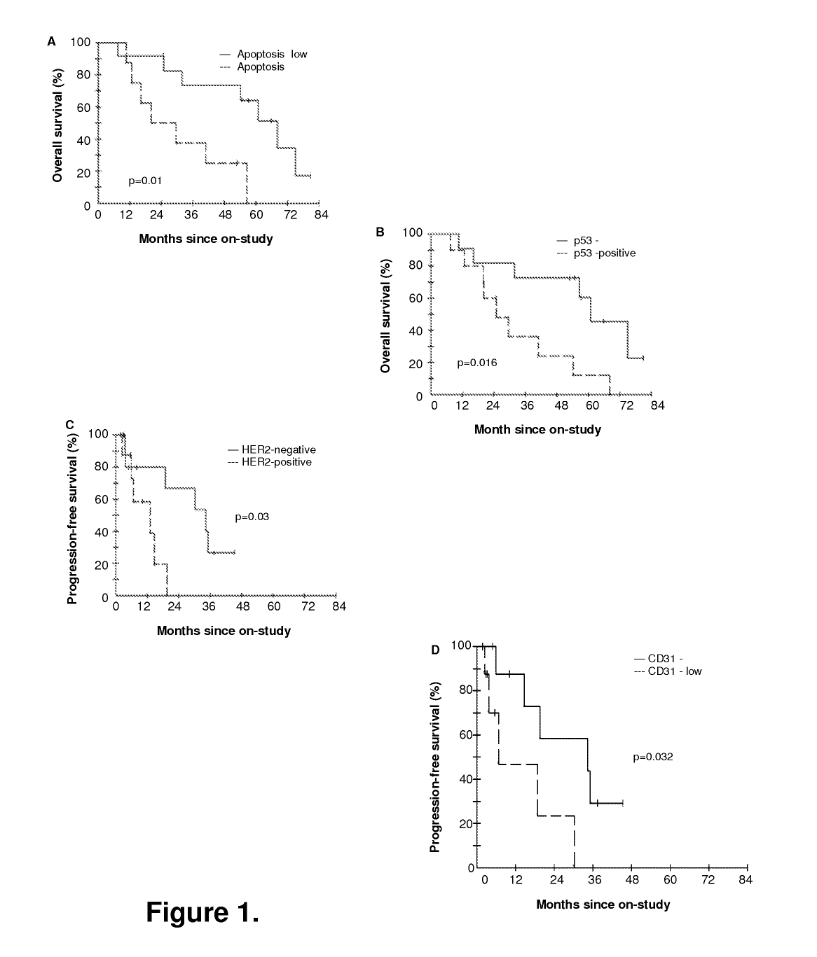

In patients treated with bevacizumab and docetaxel-doxorubicin chemotherapy, the HR for the likelihood of survival among patients with tumors containing low levels of apoptosis versus those containing high levels of apoptosis was 0.22 (p=0.011 by the log-rank test; Table 2). The HR among patients with p53-negative tumors compared with p53-positive tumors was 0.27 (p=0.016; Table 2). Patients with low baseline apoptosis or p53-negative tumors had significantly longer OS than those with high baseline apoptosis or p53-positive tumors (median 61.5 vs. 20.2 months; median 59.6 vs. 24.2 months; Table 2; FIG. 1A and 1B). (Abbreviations: ER, estrogen receptor, HER2, human epidermal growth factor receptor 2, MVD, microvessel density, No, number, PDGFR, platelet-derived growth factor receptor; pVEGFR2, phosphorylated vascular endothelial growth factor receptor 2, pVEGF, plasma vascular endothelial growth factor, sVCAM-1, vascular cell adhesion molecule-1; VEGF-A, vascular endothelial growth factor A.)

TABLE 2
Overall survival by univariate analysis.

| | Months | | |
|---|---|---|---|
| Variable | (median) | Hazard ratio (95% CI) | P-value |
| Age, years | 55.0 vs. 24.9 | 0.763 (0.273-2.134) | 0.60 |
| ≧50 vs. <50 | | | |
| Stage | 55.8 vs. 14.5 | 0.351 (0.124-0.995) | 0.041 |
| IIIA + IIIB vs. IIIC + IV | | | |
| p53 | 59.6 vs. 24.2 | 0.270 (0.087-0.837) | 0.016 |
| Negative vs. Positive | | | |
| HER2 | 54.3 vs. 26.7 | 0.602 (0.212-1.708) | 0.34 |
| Negative vs. Positive | | | |
| ER | 45.3 vs. 34.1 | 0.655 (0.207-2.076) | 0.47 |
| Positive vs. Negative | | | |
| VEGF-A | 55.4 vs. 31.8 | 0.956 (0.324-2.825) | 0.94 |
| High vs. Low | | | |
| pVEGF | 57.0 vs. 26.0 | 0.507 (0.155-1.663) | 0.25 |
| High vs. Low | | | |
| pVEGFR2-Y951 | 52.1 vs. 31.8 | 0.601 (0.185-1.955) | 0.39 |
| High vs. Low | | | |
| MVD | 41.8 vs. 38.1 | 0.822 (0.258-2.616) | 0.74 |
| Low vs. High | | | |
| Ki67 | 57.1 vs. 17.3 | 0.344 (0.103-1.150) | 0.07 |
| Low vs. High | | | |
| Apoptosis | 61.5 vs. 20.2 | 0.219 (0.061-0.781) | 0.011 |
| Low vs. High | | | |
| PDGFR-beta | 56.0 vs. 41.7 | 0.911 (0.291-2.851) | 0.87 |
| High vs. Low | | | |
| CD31 | 55.4 vs. 32.5 | 0.542 (0.159-1.857) | 0.32 |
| High vs. Low | | | |
| sVCAM-1 | 52.3 vs. 47.0 | 1.059 (0.338-3.313) | 0.92 |
| High vs. Low | | | |

Patients with low tumor Ki67 versus high Ki67 exhibited a trend towards association with a longer survival (median 57.1 vs. 17.3 months; p=0.07; Table 2). In addition, OS in patients with stage IIIA and IIIB was significantly longer than those with stage IIIC and stage IV (median 55.8 vs. 14.5 months; p=0.04). However, HER2, VEGF-A, pVEGF, ER, MVD, and VEGFR2(Y951) as well as age were not significantly associated with OS (Table 2). Although not approaching statistical significance, high baseline plasma VEGF, pVEGFR-Y951, CD31, ER-positive and HER2-negative were in the direction associated with longer survival.

Ki67 and apoptosis remained in the multivariable Cox proportional hazards model after eliminating other factors by a backward selection algorithm. When considered jointly, the HRs for the likelihood of survival among patients with low tumor apoptosis and Ki67 compared with those with high tumor apoptosis and Ki67 were 0.075 (p=0.004) and 0.098 (p=0.008), respectively. (Table 3, Abbreviations: HER2, human epidermal growth factor receptor 2, OS, overall survival, PFS, progression-free survival. a) Shown are the final models by backward selection.)

TABLE 3
OS and PFS by multivariable Cox proportional
hazards regression analysis.^{a}

| Endpoint/variable | Parameter estimate | Hazard ratio (95% CI) | P-value |
|---|---|---|---|
| OS | | | |
| Apoptosis | −2.59 | 0.075 (0.01-0.43) | 0.004 |
| Ki67 | −2.33 | 0.098 (0.02-0.55) | 0.008 |
| PFS | | | |
| HER2 | −2.43 | 0.09 (0.01-0.57) | 0.01 |
| Apoptosis | −1.52 | 0.22 (0.05-0.96) | 0.04 |

These results show that Ki67 expression in test tumor cells from breast cancer patients is associated with overall survival by multivariate analysis. Further these results show that apoptosis in test tumor cells from these patients is also associated with both overall and progression free survival by multivariate analysis.

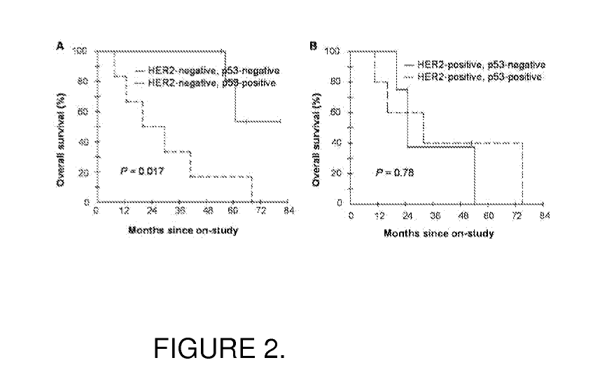

An exploratory subset analysis for survival was performed comparing patients with p53-negative tumors to those with p53-positive tumors by stratifying according to HER2 status, given the well-established roles for HER2 and p53 in the regulation of tumor angiogenesis. In patients with HER2-negative tumors, those with p53-negative tumors survived significantly longer than those with p53-positive tumors (median not reached vs. 20 months; p=0.017; FIG. 2A). In contrast, among patients with HER2-positive tumors, OS was similar in both p53-negative tumors and p53-positive tumors (median 20.0 vs. 23.4 months; p=0.78; FIG. 2B).

### Marker Status and Progression Free Survival.

The status of the characteristics of the tumor sample was assessed in relationship to disease progression. Table 4 shows the HRs for the risk of progression according to the status of certain characteristics of the tumor samples. (Abbreviations: ER, estrogen receptor, HER2, human epidermal growth factor receptor 2, MVD, microvessel density, No, number, PDGFR, platelet-derived growth factor receptor; pVEGFR2, phosphorylated vascular endothelial growth factor receptor 2, pVEGF, plasma vascular endothelial growth factor, sVCAM-1, vascular cell adhesion molecule-1; VEGF-A, vascular endothelial growth factor A.)

TABLE 4
Progression-free survival by univariate analysis.

| | Months | | |
|---|---|---|---|
| Variable | (median) | Hazard ratio (95% CI) | P-value |
| Age, years | 14.2 vs. 18.8 | 0.857 (0.278-2.710) | 0.79 |
| ≧50 vs. <50 | | | |
| Stage | 19.0 vs. 6.4 | 0.561 (0.156-2.014) | 0.37 |
| IIIA + IIIB vs. IIIC + IV | | | |
| p53 | 21.0 vs. 16.1 | 0.586 (0.185-1.858) | 0.36 |
| Negative vs. Positive | | | |
| HER2 | 31.2 vs. 9.4 | 0.227 (0.054-0.960) | 0.03 |
| Negative vs. Positive | | | |
| ER | 30.2 vs. 13.7 | 0.232 (0.030-1.811) | 0.13 |
| Positive vs. Negative | | | |
| VEGF-A | 16.3 vs. 18.3 | 0.682 (0.204-2.283) | 0.53 |
| High vs. Low | | | |
| pVEGF | 12.5 vs. 15.0 | 0.5961 (0.279-3.305) | 0.95 |
| High vs. Low | | | |
| pVEGFR2-Y951 | 24.0 vs. 14.3 | 0.620 (0.186-2.071) | 0.43 |
| High vs. Low | | | |
| MVD | 16.8 vs. 16.2 | 0.854 (0.246-2.964) | 0.80 |
| Low vs. High | | | |
| Ki67 | 22.7 vs. 11.2 | 0.767 (0.222-2.642) | 0.67 |
| Low vs. High | | | |
| Apoptosis | 20.7 vs. 6.4 | 0.364 (0.103-1.282) | 0.10 |
| Low vs. High | | | |
| PDGFR-beta | 20.8 vs. 19.3 | 0.513 (0.119-2.210) | 0.36 |
| High vs. Low | | | |
| CD31 | 28.0 vs. 6.2 | 0.228 (0.053-0.982) | 0.032 |
| High vs. Low | | | |
| sVCAM-1 | 11.8 vs. 26.9 | 2.855 (0.727-11.211) | 0.12 |
| High vs. Low | | | |

Patients with HER2-negative tumors had a significantly longer PFS than those with HER2-positive tumors (median 31.2 vs. 9.4 months; p=0.03; Table 3; FIG. 1C). The other angiogenic or tumor factors studied were not significantly associated with PFS by univariate analysis (Table 4). By multivariable Cox proportional hazards regression analysis using a backward selection algorithm, HER2 and apoptosis were significantly associated with progression when considered jointly in the model. The HRs for progression in HER2-negative tumors compared with HER2-positive tumors was 0.088 (p=0.01), and in low apoptosis versus high apoptosis was 0.22 (p=0.04; Table 3).

Tumor and angiogenic factors that play important roles in angiogenesis correlated with tumor progression, and clinical outcome. The results demonstrate that baseline p53 and apoptosis are significantly associated with OS by univariate analyses in patients who received neoadjuvant bevacizumab followed by bevacizumab plus doxorubicin-docetaxel chemotherapy. The data are substantiated by preclinical findings. p53 mediates inhibition of tumor angiogenesis through multiple mechanisms such as degradation of HIF-1 alpha, up-regulation of a collagen prolyl hydroxylase, which releases the anti-angiogenic fragments of collagen type 4 and 18 to the extracelluar matrix, and induction of microRNA-107 that inhibits HIF-1. On the other hand, p53 dysfunction has been associated with the increase in VEGF expression and decrease in expression of thrombospondin-1, a potent inhibitor of angiogenesis or neovascularization. In addition, p53-deficient animal models were less responsive than wild-type p53 models to anti-angiogenic and cytotoxic combination therapy. In a large study, node-positive breast cancer patients received adjuvant fluorouracil-doxorubicin-cyclophosphamide or docetaxel-doxorubicin-cyclophosphamide chemotherapy; p53 was significantly associated with both DFS and OS for 1350 patients. In this pilot trial, p53 in significant association with survival may reflect the profound and collective effects of p53 on angiogenesis and responses to chemotherapy.

The low levels of baseline tumor apoptosis relative to high levels of baseline tumor apoptosis may represent a status of adequate versus inadequate tumor angiogenesis. The tumors with adequate angiogenesis status reflected by low levels of tumor apoptosis may be more sensitive to anti-angiogenesis therapy, and thus was associated with outcome in patients after bevacizumab in combination with chemotherapy. Moreover, baseline tumor apoptosis and proliferation were found to be jointly and independently associated with the treatment outcome after multivariable modeling which potentially took into account the classical prognostic factors. Patients with tumors with low intrinsic proliferation rate, possibly representative of a subset of patients with relatively indolent tumors, and baseline adequate tumor angiogenesis status (low levels of apoptosis index) identify subgroups of patients with significantly longer survival after bevacizumab anti-angiogenic therapy plus chemotherapy. In addition, patients with HER2-negative tumors had a significantly longer PFS than those with HER-positive tumors. HER2 and apoptosis are independently and jointly associated with disease progression as demonstrated by multivariable analysis. In previous preclinical and clinical studies, it has been shown that HER2 associates with disease progression, and increases angiogenesis in breast cancer. The other markers examined were not significantly associated with PFS in this cohort.

However, there were no significant associations between some critical angiogenic factors (VEGF-A, pVEGF, VEGFR2-Y951, and MVD) and PFS. Instead, as discussed above, some critical tumor factors were found to be significantly associated with the treatment outcome. However, it is of importance to mention that although not approaching statistical significance, tumor pVEGFR-Y951, PDGFR-beta, ER-negative and p53-negative were in the direction associated with longer PFS. In contrast, high baseline serum VCAM-1 was in the direction associated with a shorter PFS.

Since bevacizumab was initially approved for use in HER2-negative metastatic breast cancer, and the facts that HER2 is associated with PFS and p53 is significant for OS by univariate analyses in this study, an exploratory subset analysis of survival was performed comparing patients with p53-negative tumors to those with p53-positive tumors by stratifying according to HER2 status. Patients with tumors that were both HER2-negative and p53-negative survived significantly longer than those with tumors that were HER2-negative and p53-positive after the combination treatment (FIG. 2A). p53 negativity, possibly functioning as a negative regulator of tumor angiogenesis and as a tumor suppressor, and the lack of HER2 overexpression, a positive regulator of angiogenesis and tumor progression, are critical for survival in patients who received bevacizumab plus chemotherapy. However, this data is considered to be hypothesis-generating due to the small sample size, and warrants further validation by other studies. In a Phase III study, the addition of bevacizumab to first-line irinotecan, 5-flurouracil, and leucovorin (IFL) prolonged median survival in patients with metastatic colorectal cancer (Hurwitz H, Fehrenbacher L, Novotny W, et al.: Bevacizumab plus irinotecan, fluorouracil, and leucovorin for metastatic colorectal cancer. N Engl J Med 350: 2335-42, 2004). With a retrospective analysis of the status of certain characteristics of the tumor in association with the treatment effect of bevacizumab, patients with p53 protein-negative tumors had a prolonged survival in patients treated with IFL plus bevacizumab as compared with those treated IFL plus placebo (median 25.07 vs. 16.26 months).

The disclosure of all publications cited to in this description are hereby incorporated by such reference in their entirety.

It will be apparent that the precise details of the methods described herein may be varied or modified without departing from the spirit of the disclosed invention. We claim all modifications and variations that fall within the scope and spirit of the claims below.

1. A method for identifying a cancer patient for longer overall survival that is amenable to a combination of anti-angiogenesis therapy and chemotherapy, comprising measuring baseline p53 expression and apoptosis in a tumor sample from said cancer patient, whereby negative p53 and low percentage of tumor apoptosis before therapy indicate that the cancer patient will be amenable to the combination therapy.

2. A method for identifying a cancer patient that is not amenable to the combination of anti-angiogenesis therapy and chemotherapy, comprising measuring baseline p53 expression and apoptosis in a tumor sample from said cancer patient, whereby positive p53 and high levels of tumor apoptosis indicate that the cancer patient will not be amenable to the combination therapy due to shorter overall survival.

3. The method of claim 1, wherein the anti-angiogenesis therapy comprises administration of a therapeutic agent selected from the group consisting of an anti-vascular endothelial growth factor (VEGF) monoclonal antibody, and a small-molecule kinase inhibitor that inhibits activation of a VEGF receptor, Raf, a platelet-derived growth factor receptor (PDGFR), or Kit.

4. The method of claim 3, wherein the therapeutic agent is bevacizumab, sunitinib, sorafenib, or pazopanib.

5. The method of claim 1, wherein the anti-angiogenesis therapy comprises administration of bevacizumab.

6. The method of claim 5, wherein the combination therapy further comprises administration of a chemotherapeutic agent selected from the drug classes consisting of a taxane (paclitaxel or docetaxel), an anthracycline (doxorubicin or epirubicin), cyclophosphamide, capecitabine, tamoxifen, letrozole, carboplatin, gemcitabine, cisplatin, erlotinib, irinotecan, fluorouracil, and oxaliplatin.

7. The method of claim 6 wherein the cancer patient is selected from the group of cancer patients having solid tumors derived from breast, cervical, prostate, ovary, renal carcinoma, lung, gastric, pancreas, glioblastoma, and colorectal.

8. The method of claim 7, wherein the cancer patient has a solid tumor derived from breast.

9. The method of claim 8, wherein a capecitabine-based therapy is administered.

10. The method of claim 8, wherein a paclitaxel-based therapy is administered.

11. The method of claim 8, wherein an anthracycline-based therapy is administered.

12. The method of claim 8, wherein a docetaxel-based therapy is administered.

13. The method of claim 8, further comprising measuring HER2 expression or HER2 gene amplification in a tumor sample of the patient.

14. The method of claim 13, further comprising measuring the percentage of apoptosis in a tumor sample of the patient.

15. The method of claim 14, wherein the percentage of apoptosis in the tumor sample is low.

16. The method of claim 13, further comprising measuring the percentage of p53 expression in the tumor sample.

17. The method of claim 13, wherein the patient is identified based on the percentage of p53 expression and level of HER2 expression in the tumor sample.

18. The method of claim 17, wherein the percentage of p53 expression and level of HER2 expression or HER2 gene amplification, or dual p53 HER2 expression in the tumor sample are negative and endothelial cell CD31 expression is high in the tumor sample.

19. A method for identifying a breast cancer patient for longer overall survival or progression-free survival resulting from anti-angiogenesis therapy, comprising measuring the expression of certain characteristic factors of a tumor sample of said patient, and identifying said patient for anti-angiogenic treatment based on the expression of the characteristic factor in the tumor sample, wherein the anti-angiogenic therapy consists of neoadjuvant bevacizumab plus chemotherapy.

20. The method of claim 19, wherein the factors are of the tumor cell expression of p53, HER2, HER2 gene amplification, or percentage of apoptosis or endothelial cell CD31 expression in the tumor sample.

21. The method of claim 18, wherein the factors are tumor cell expression of p53, HER2, or HER2 gene amplification, or percentage of apoptosis or endothelial cell CD31 expression in the tumor sample..

22. The method of claim 19, wherein the chemotherapy is docetaxel/doxorubicin-based.

23. The method of claim 18, wherein the chemotherapy is paclitaxel-based.

24. A method for identifying a breast cancer patient for longer overall survival or progression-free survival resulting from neoadjuvant antiangiogenesis therapy plus chemotherapy, comprising measuring tumor cell expression of p53, HER2,or HER2 gene amplification, or percentage of apoptosis or endothelial cell CD31 expression in the tumor sample, wherein the cancer of the breast cancer patient is a locally advanced breast cancer, an early stage breast cancer, or an inflammatory breast cancer before surgery, and wherein antiangiogenic therapy comprises administration of bevacizumab before surgery.

25. A method for identifying a breast cancer patient for antiangiogenesis therapy plus chemotherapy for the management metastatic or locally recurrent disease, comprising measuring tumor cell expression of p53, HER2, or HER2 gene amplification, or percentage of apoptosis or endothelial cell CD31 expression in the tumor sample, wherein the cancer of the breast cancer patient is a locally recurrent breast cancer or a metastatic breast cancer, and wherein antiangiogenic therapy comprises administration of bevacizumab.

26. A method for identifying a breast cancer patient for adjuvant antiangiogenesis therapy plus chemotherapy, comprising measuring tumor cell expression of p53, HER2, or HER2 gene amplification, or percentage of apoptosis or endothelial cell CD31 expression in the tumor sample, wherein the cancer is an early operable breast cancer, and wherein antiangiogenic therapy comprises administration of bevacizumab after surgery.

27. A method of diagnosing a tumor in a subject, the method comprising:
a) providing a test tumor cell population from a tumor sample of the subject prior to therapy;
b) measuring the percentage of p53-positive cells and apoptotic cells in the test cell population; wherein a low percentage of p53-positive cells and apoptotic cells indicates that the tumor is amenable to a combination of anti-angiogenesis therapy and chemotherapy for longer overall survival or progression-free survival of the subject.

28. The method of claim 27, further comprising a step of measuring the level of HER2 or HER2 gene amplification in the test cell population, wherein a low percentage of p53-positive cells, low percentage of apoptotic cells and low level of HER2 and high level of endothelial CD31 expression indicates that the tumor is amenable to a combination of anti-angiogenesis therapy and chemotherapy for better treatment outcome.

29. The method of claim 27, wherein the test endothelial cell population stained for CD31 is scored as high if the staining index >25.

30. The method of claim 28, wherein the tumor is determined to be amenable to the combination therapy for longer overall survival or progression-free survival when the test cell population shows the tumor cells are p53-negative, are HER2-negative, are dual p53 HER2-negative, have low percentages apoptosis, and are high for endothelial cell CD31 expression.

31. The method of claim 27, wherein less than 10% of the test cell population stained for p53 is defined as p53-negative.

32. The method of claim 27, wherein less than or equal to 2% of the test cell population labeled for apoptosis is defined as low.

33. The method of claim 27, wherein a scoring of 0 or 1+ for HER2 or 2+ when HER2 gene is non-amplified in the test cell population is defined as HER2-negative.

34. The method of claim 30, wherein the anti-angiogenesis therapy comprises administration of a therapeutic agent selected from the group consisting of an anti-vascular endothelial growth factor (VEGF) monoclonal antibody, and a small-molecule kinase inhibitor that inhibits activation of VEGF receptor, Raf, platelet-derived growth factor receptor (PDGFR), or Kit.

35. The method of claim 30, wherein the anti-angiogenesis therapy comprises administration of bevacizumab.

36. The method of claim 30, wherein the chemotherapy comprises administration of a chemotherapeutic agent selected from the drug classes consisting of paclitaxel, docetaxel, an anthracycline (doxorubicin or epirubicin), cyclophosphamide, capecitabine, tamoxifen, letrozole, carboplatin, gemcitabine, cisplatin, erlotinib, irinotecan, fluorouracil, and oxaliplatin.

37. The method of claim 36, wherein the cancer patient is selected from the group of cancer patients having solid tumors derived from breast, cervical, prostate, ovary, renal carcinoma, lung, gastric, pancreas, glioblastoma, and colorectal.

38. The method of claim 37, wherein the cancer patient has a solid tumor derived from breast.

39. The method of claim 38, wherein a capecitabine-based therapy is administered.

40. The method of claim 38, wherein a paclitaxel-based therapy is administered.

41. The method of claim 38, wherein an anthracycline-based therapy is administered.

42. The method of claim 38, wherein a docetaxel-based therapy is administered.
